(19) United States
(12) Patent Application Publication
(10) Pub. No.: US 2015/0182226 A1
(43) Pub. Date: Jul. 2, 2015

(54) VASO-OCCLUSIVE COIL WITH BENDING SECTIONS

(71) Applicants: STRYKER CORPORATION, Kalamazoo, MI (US); STRYKER EUROPEAN HOLDINGS I, LLC, Kalamazoo, MI (US)

(72) Inventor: Clifford Teoh, Los Altos, CA (US)

(21) Appl. No.: 14/580,713

(22) Filed: Dec. 23, 2014

Related U.S. Application Data
(60) Provisional application No. 61/920,893, filed on Dec. 26, 2013.

(51) Int. Cl. A61B 17/12

(52) U.S. Cl. CPC A61B 17/12113; A61B 17/1214; A61B 17/12031

(57) ABSTRACT

A vaso-occlusive device is formed from an elongate strand of material wound by a winding process into a helical coil having a length, wherein one or more discrete sections of the coil along its length were subjected to less mechanical stress or strain than a remainder of the coil during the winding process, such that each of the one or more discrete sections has a lower stiffness and greater flexibility than a respective stiffness and flexibility of the remainder of the coil, and such that each of the discrete one or more sections acts as a bending location along the length of the coil when the coil is delivered into an aneurysm.

### RELATED APPLICATION DATA

The present application claims the benefit under 35 U.S.C. §119 to U.S. provisional patent application Ser. No. 61/920,893, filed Dec. 26, 2013. The foregoing application is hereby incorporated by reference into the present application in its entirety.

### FIELD

The field of the disclosed inventions generally relates to vaso-occlusive devices for establishing an embolus or vascular occlusion in a vessel of a human or veterinary patient. More particularly, the disclosed inventions relate to vaso-occlusive coils having manufactured bending sections along their length.

### BACKGROUND

Vaso-occlusive devices or implants are used for a wide variety of reasons, including treatment of intra-vascular aneurysms. Commonly used vaso-occlusive devices include soft, helically wound coils formed by winding a platinum (or platinum alloy) wire strand or filament about a “primary” mandrel. The coil is then wrapped around a larger, “secondary” mandrel, and heat treated to impart a secondary shape. For example, U.S. Pat. No. 4,994,069, issued to Ritchart et al., which is fully incorporated herein by reference, describes a vaso-occlusive device that assumes a linear, helical primary shape when stretched for placement through the lumen of a delivery catheter, and a folded, convoluted secondary shape when released from the delivery catheter and deposited in the vasculature. Wound coils may also be formed using the point winding or “mandreless” method. In the mandreless coil winding method, a wire strand or filament is wound onto a deflection surface.

In order to deliver the vaso-occlusive devices to a desired site in the vasculature, e.g., within an aneurysmal sac, it is well-known to first position a small profile, delivery catheter or “micro-catheter” at the site using a steerable guidewire. Typically, the distal end of the micro-catheter is provided, either by the attending physician or by the manufacturer, with a selected pre-shaped bend, e.g., 45°, 26°, “J”, “S”, or other bending shape, depending on the particular anatomy of the patient, so that it will stay in a desired position for releasing one or more vaso-occlusive device(s) into the aneurysm once the guidewire is withdrawn. A delivery or “pusher” wire is then passed through the micro-catheter, until a vaso-occlusive device coupled to a distal end of the delivery wire is extended out of the distal end opening of the micro-catheter and into the aneurysm. Once in the aneurysm, the vaso-occlusive devices deform or bend to allow packing The vaso-occlusive device is then released or detached from the end delivery wire, and the delivery wire is withdrawn back through the catheter. Depending on the particular needs of the patient, one or more additional occlusive devices may be pushed through the catheter and released at the same site.

One well-known way to release a vaso-occlusive device from the end of the pusher wire is through the use of an electrolytically severable junction, which is a small exposed section or detachment zone located along a distal end portion of the pusher wire. The detachment zone is typically made of stainless steel and is located just proximal of the vaso-occlusive device. An electrolytically severable junction is susceptible to electrolysis and disintegrates when the pusher wire is electrically charged in the presence of an ionic solution, such as blood or other bodily fluids. Thus, once the detachment zone exits out of the catheter distal end and is exposed in the vessel blood pool of the patient, a current applied through an electrical contact to the conductive pusher wire completes an electrolytic detachment circuit with a return electrode, and the detachment zone disintegrates due to electrolysis.

In order to better frame and fill aneurysms, it is well-known to impart complex three-dimensional secondary shapes on vaso-occlusive coils. However, imparting a secondary shape on a coil increases the contact force imparted by the coil on the inner surface of the microcatheter during delivery. This additional contact force increases the friction between the coil and the microcatheter, interfering with delivery of the coil. Another perceived problem of vaso-occlusive coils with secondary structures is rotation of the distal portion of the coil immediately after it exits out the open end of the microcatheter. This complicates targeting during delivery.

In the field of vaso-occlusive coils, secondary shape and coil stiffness, flexibility, and softness affect the ability of a coil to frame and distribute itself within an aneurysm. Many secondary shapes and stiffnesses are known. One example is a coil with a primary main coil having a twisting triangular cross-section and a boxed shaped secondary structure. While the twisting triangular shape of the primary coil does impart areas of preferential folding of the coil along the sides of the triangle, it limits the number of flex transition points as the coil will resist folding at the apex of the twisting triangle (compared to the side of the twisting triangle). Accordingly, there exists a need for a vaso-occlusive coil with flex points that can be located anywhere over a substantial portion of the primary coil winding without requiring (or otherwise minimizing) additional preset secondary shape.

### SUMMARY

In accordance with one embodiment of the disclosed inventions, a vaso-occlusive device is formed from an elongate strand of material wound by a winding process into a helical coil having a length, wherein one or more sections of the coil along its length were subjected to less mechanical stress or strain than a remainder of the coil during the winding process, such that each of the one or more sections has a lower stiffness and greater flexibility than a respective stiffness and flexibility of the remainder of the coil, and such that each of the one or more sections acts as a bending location along the length of the coil when the coil is delivered into an aneurysm.

By way of example, each of the one or more sections of the coil can be subjected to less mechanical stress or strain than the rest of the coil during the winding process by one or both of (i) a temporary decrease in a tension imparted on the elongate strand during winding of the respective discrete section, and (ii) a temporary increase in an angle formed by the elongate strand and a mandrel on which the coil was wound during winding of the respective discrete section. However formed, the resulting one or more “bending” sections of the coil preferably have substantially same dimensions as the remainder of the coil, at least as can be detected without using any visual aid. In particular, the windings of the one or more bending sections of the coil preferably have a substantially same pitch as windings of the remainder of the coil.

Particularly applicable to a relatively short coil, the one or more bending sections may consist of a single bending section along the length of the coil. In other embodiments, applicable to longer coils, the one or more bending sections include multiple bending sections along the length of the coil, which may be substantially uniform in length, or which may vary in length. The transitions between respective non-bending sections and bending sections may be gradual or abrupt (as defined herein), or some of each.

In accordance with another embodiment of the disclosed inventions, a vaso-occlusive device is formed from an elongate strand of material wound by a winding process into a helical coil, the coil comprising a plurality of bending sections and a plurality of non-bending sections, wherein individual bending sections alternate with individual non-bending sections along a length of the coil, and wherein the bending sections were subjected to less mechanical stress or strain than the non-bending sections during the winding process, such that the bending sections have a lower stiffness and greater flexibility than a respective stiffness and flexibility of the non-bending sections.

By way of example, the bending sections can be subjected to less mechanical stress or strain than the non-bending sections during the winding process due to one or both of (1) a temporary decrease in a tension imparted on the elongate strand during winding of each bending section, and (ii) a temporary increase in an angle formed by the elongate strand and a mandrel on which the coil was wound during winding of each bending section. However formed, the bending sections preferably have substantially same dimensions as the non-bending sections, in particular, wherein windings of the bending sections have a substantially same pitch as windings of the non-bending sections.

In various embodiments, the respective bending sections may be substantially uniform in length. In some embodiments, the non-bending sections will decrease in length from a distal end of the coil to a proximal end of the coil. Again, transitions between respective non-bending sections and bending sections may be gradual or abrupt, or some of each.

Other and further aspects and features of embodiments of the disclosed inventions will become apparent from the ensuing detailed description in view of the accompanying figures.

### BRIEF DESCRIPTION OF THE DRAWINGS

The drawings illustrate the design and utility of embodiments of the disclosed inventions, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments and are not therefore to be considered limiting of its scope.

FIG. 1 is a detailed longitudinal cross-section view of a vaso-occlusive device constructed according to one embodiment of the disclosed inventions.

FIG. 2 is a side view of a vaso-occlusive device constructed according to another embodiment of the disclosed inventions.

FIGS. 3A to 3C are detailed longitudinal cross-section views of a vaso-occlusive device constructed according to yet another embodiment of the disclosed inventions, as the vaso-occlusive device is delivered from a catheter into a substantially closed space (e.g., an aneurysm).

FIGS. 4 and 5 are schematic views of a vaso-occlusive coil being wound according to one embodiment of the disclosed inventions.

FIGS. 6A and 6B are side views of two vaso-occlusive coils wound according respective embodiments of the disclosed inventions. The coils are partially supported on a higher surface and partially suspended over a lower surface.

FIG. 7 is a perspective view of a vaso-occlusive device in a natural state mode, illustrating one exemplary secondary configuration according to an embodiment of the disclosed inventions.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, he terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. As used in this specification and the appended claims, the term “strand” includes, without limitation, the terms “wire,” “filament,” “fiber,” and the like. As used in this specification and the appended claims, the term “dimension” includes, without limitation, strand thickness, coil outer diameter, inner coil lumen diameter and coil winding pitch. As used in this specification and the appended claims, the phrase “abrupt changes in a coil,” and its equivalents, mean changes that occur over 1 to 3 windings of the coil. As used in this specification and the appended claims, the phrase “gradual changes in a coil,” and its equivalents, mean changes that occur over 4 windings to the entire length of a coil segment.

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

This disclosure describes methods for manufacturing a helically wound coil, including modifying both primary and secondary winding parameters, such as filament tension, filament angle, and mandrel tension, to produce a primary wound coil having alternating stiffer and softer segments along its length. In particular, a coil produced according to the disclosed processes can have segments along its length that vary in stiffness, flexibility, and/or softness, while the coil retains substantially similar dimensions throughout its length, i.e., such that there are no perceptible differences to the unaided eye. In particular, periodic softer segments having a greater flexibility (less stiffness) than the rest of the coil interspersed along the length of the coil are configured to create bend/flex points in the coil to provide for better framing and/or filling of an aneurysm, without having to further impart any secondary shape on the primary wound coil. In some embodiments, it may be desirable to still impart some secondary shape features, such as a preset shape on the distal end, e.g., a “J” shape or 45 degree angle, to direct the coil more centrally, and a helical loop (“pig tail”) on proximal end of the coil to help prevent the coil tail from extending from the aneurysm into the parent vessel.

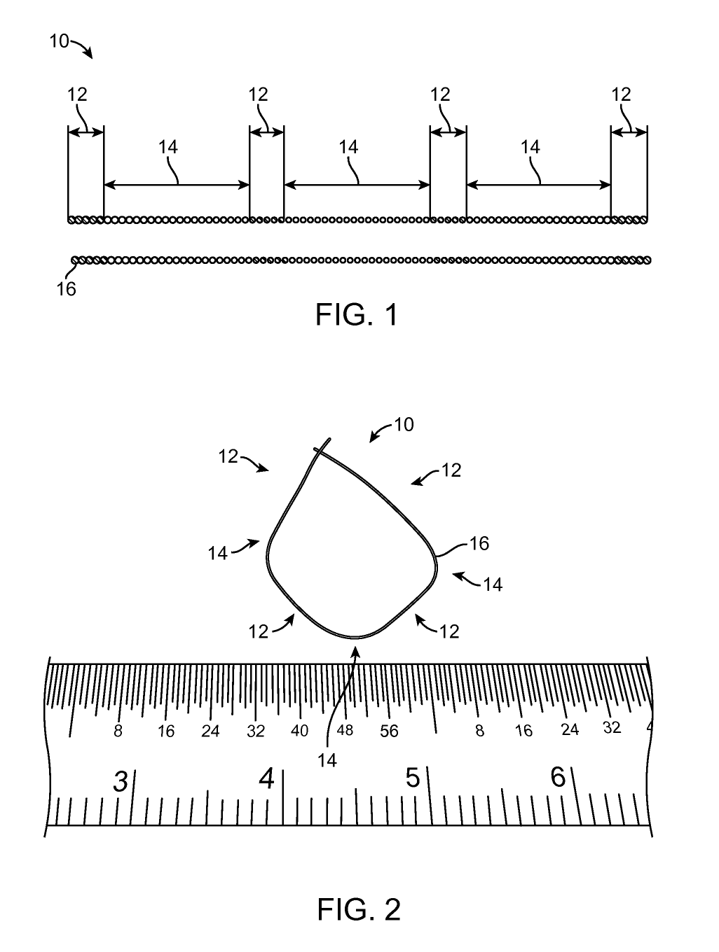

FIG. 1 illustrates a vaso-occlusive device 10 in accordance with one embodiment. The vaso-occlusive device 10 is formed by a single filament 16 that has been wound into a helical primary coil having a length. The vaso-occlusive coil 10 has alternating stiffer segments 12 and softer (or “bending”) segments 14 along its length, each of the individual stiffer and softer segments 12, 14 including a plurality of coil windings.

The filament 16 is made from the any suitable biocompatible material. For example, filament 16 may be made from a metal, such as pure platinum. In other embodiments, the filament 16 may be made from an alloy, such as platinum-tungsten alloy, e.g., 8% tungsten and the remainder platinum. In further embodiments, the filament 16 may be made from platinum-iridium alloy, platinum rhenium alloy, or platinum palladium alloy. In still other embodiments, the filament 16 may be made from a platinum core with an outer layer of platinum-tungsten alloy, or from a material consisting of a core of platinum-tungsten alloy and an outer layer of platinum. In yet other embodiments, the filament 16 may be made from biopolymers bioactive material, or a combination of such materials. For example, a bioactive coating may be applied to any of the metallic, and/or biopolymeric filaments 16 described above. Examples of polymers from which filaments 16 may be made include polypropylene, polyethylene, poly propylene/ethylene copolymer, nylon, polyester, PVDF, and PTFE.

It should be appreciated that the filament materials not be limited to the examples described above. In any of the embodiments described herein, the filament material may be a radio-opaque material such as a metal or a polymer. Also, in other embodiments, the filament material may be rhodium, palladium, rhenium, as well as tungsten, gold, silver, tantalum, and alloys of these metals. These metals have significant radio-opacity and in their alloys may be tailored to accomplish an appropriate blend of flexibility and stiffness. They are also largely biologically inert.

The filament material may be any materials which maintain their shape despite being subjected to high stress may be used to construct the coils. For example, certain “super-elastic alloys” include various nickel/titanium alloys (48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum), may be used. In further embodiments, titanium-nickel alloy known as “nitinol” may be used to form the filaments 16. These are very sturdy alloys which will tolerate significant flexing without deformation even when used as very small diameter wire.

In the vaso-occlusive coil 10 depicted in FIG. 1, each stiffer segment 12 measures 0.5 mm and each softer segment 14 measures 2.0 mm. Of course, the length of the filament 16 forming the windings of the stiffer and softer segments 12, 14 is greater than the length the stiffer and softer segments 12, 14, with the length of filament 16 depending on the diameter of the coil 10. While the stiffer and softer segments 12, 14 depicted in FIG. 1 are 0.5 mm and 2.0 mm long, respectively, other lengths of stiffer and softer segments 12, 14 are within the scope of the claims. Lengths of stiffer and softer segments 12, 14 may be uniform along the vaso-occlusive coil 10, or the lengths of various segments 12, 14 may vary along length of vaso-occlusive coil 10. Each of the alternating stiffer and softer segments 12, 14 may be the same or different lengths when the vaso-occlusive coil is in its primary shape. The changes between stiffer and softer segments 12, 14 can be gradual or abrupt transitions. For example, winding parameters can be adjusted gradually, instead of abruptly, for a smoother transition in stiffness. For instance, the stiffer and softer segments 12, 14 can each be 5 mm long, as in the vaso-occlusive coil 10 depicted in FIG. 2.

FIG. 2 depicts a vaso-occlusive coil 10 having 4 stiffer segments 12 and 3 softer segments 14 that are interleaved along the vaso-occlusive coil 10. The stiffer segments 12 are straight, and the softer segments 14, which span several dozen windings, act as bending locations in the coil, and are bent or curved to allow the coil 10 to loop into an approximately circular shape with apices at the softer segments 14. The vaso-occlusive coil 10 has been forced into this approximately circular shape by being introduced into a substantially closed space (not shown, simulating an aneurysm) having a single opening. The substantially closed space (not shown) has an inner dimension approximately equal to the length of the stiffer segment 12 (0.5 mm). Therefore, when the distal end of the vaso-occlusive coil 10 is introduced into the substantially closed space, an inner wall of the substantially closed space exerts pressure on the vaso-occlusive coil 10 after the first stiffer segment 12 is deployed from the catheter and during deployment of the first softer segment 14. The exerted pressure causes the vaso-occlusive coil 10 to deform/bend at the first softer segment 14 in a random fashion. This cycle is repeated over the next two stiffer segment 12 and two softer segments 14, resulting in the shape depicted in FIG. 2. The pressure exerted by the inner walls of the substantially closed space has bent, at the softer segments 14, the vaso-occlusive coil 10 into an approximately circular shape with apices.

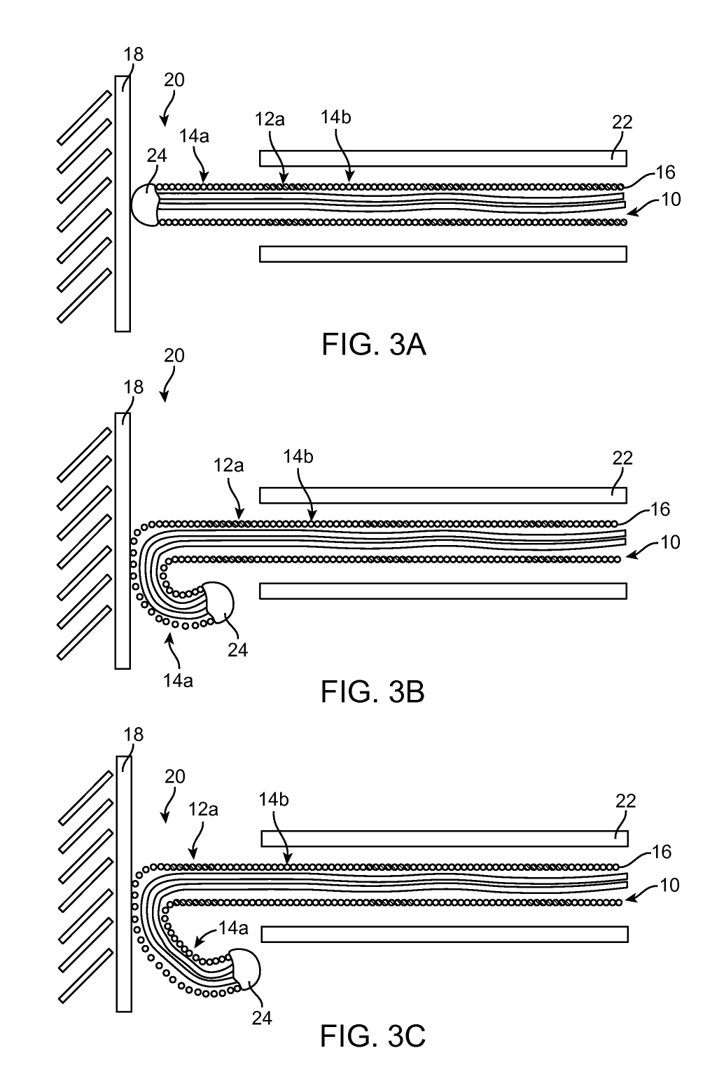

FIGS. 3A to 3C depict a vaso-occlusive device 10 according to another embodiment as it is delivered from a catheter 22 into a substantially closed space 20, like an aneurysm. The vaso-occlusive coil 10 has a series of alternating 2.0 mm softer segments 14 and 0.75 mm stiffer segments 12, as shown in FIG. 3A. This configuration may be suited for fill/finish embolization of a 3-5 mm aneurysm.

These figures illustrate the interaction of a vaso-occlusive coil 10 and an inner wall 18 of a substantially closed space 20 in greater detail. Complexing, bending, or flexing of the coil 10 is based on the span of the exposed coil 10 (i.e., distance from aneurysm wall and distal tip of catheter 22) and the softness of the coil segment positioned within the span. The span of the delivery procedure depicted in FIGS. 3A to 3C is about 2 mm.

FIG. 3A depicts a vaso-occlusive coil 10 being deployed from a catheter 22 into a substantially closed space 20. The distal most (“first”) softer segment 14a is coupled to a distal end cap 24. The distal end cap 24 of the coil 10 encounters a wall (e.g., of an aneurysm) or other resistance before the first softer segment 14a completely enters the 2.0 mm span.

Once the distal end 24 contacts the inner wall 18, the unsupported first softer segment 14a begins to flex/bend/complex within the span in a random fashion as the vaso-occlusive coil 10 is fed down the catheter 22 in a distal direction for delivery, as shown in FIG. 3B. Once, this softer segment 14a deflects slightly, and the coil 10 is further deployed. The first softer segment 14a bends closer to the inner wall 18 than the catheter 22. Bending of the first softer segment 14a absorbs the distally directed delivery force exerted on the vaso-occlusive coil 10.

As shown in FIG. 3B, the distal most (“first”) stiffer segment 12a begins to exit the catheter 22. The first stiffer segment 12a pushes distally with greater column strength to facilitate further deployment, and to “seek” other open spaces, because it is a stiffer coil than the first softer segment 14a which has flexed and deflected out of the way. As the second softer segment 14b is deployed from the catheter 22, the first softer segment 14a continues to bend because it is already in the process of bending.

As the first stiffer segment 12a is fully deployed, the trailing second softer segment 14b will eventually exit the catheter 22, as shown in FIG. 3C. Once a certain length of the second softer segment 14b is deployed into the span, the second softer segment 14b will preferentially flex/bend/complex and the cycle described above repeats. When the first stiffer segment 12a contacts the inner wall 18 the bending process begins again with the second softer segment 14b. The first stiffer segment 12a does not bend when urged against the inner wall 18, so the second softer segment 14b begins to bend. The second softer segment 14b bends in a random direction unrelated to the direction in which the first softer segment 14a previously bent. This bending process is repeated with each softer segment 14 until the vaso-occlusive device 10 has been delivered into the substantially closed space 20 (e.g., aneurysm).

The vaso-occlusive coil 10 can be constructed from materials that can be visualized (e.g., the radio-opaque materials described above), to enable a user to visualize and control the coil behavior during delivery. With such control during delivery, the user can achieve the desired coil distribution and filling level by advancing and retracting the catheter 22 tip relative to the exposed coil segments and aneurysm position.

The lengths of the stiffer and softer segments 12, 14 can be modified to customize the vaso-occlusive coil 10 for particular functions and/or substantially closed spaces 20. For instance, the length of stiffer segments 12 can be matched to a half circumference of a spherical substantially closed space 20 to cause the vaso-occlusive coil 10 to “frame” the substantially closed space 20, i.e., form a three dimensional outline of the substantially closed space 20. In the alternative, the length of the softer segments 14 can be increased to cause the vaso-occlusive coil 10 to “fill” a space, i.e., form a mass of increasing size in the immediate vicinity at the distal end of the catheter 22. In another alternative, the length of the stiffer segments 12 can be increased to cause the vaso-occlusive coil 10 to “seek” open spaces inside of the substantially closed space 20. Such coils 10 will be “space seeking coils,” which have greater column strength to seek open spaces away from the distal end of the catheter 20. Thus, coil behavior can be tailored to the specific need or application.

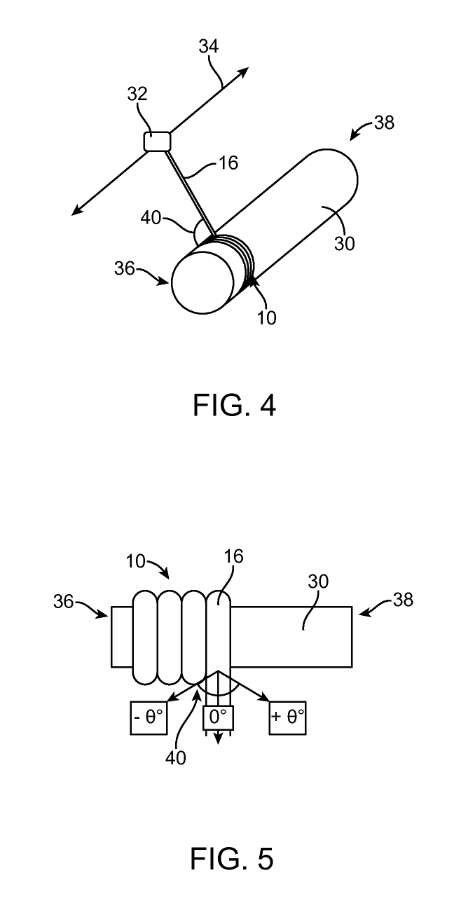

The stiffness/flexibility of the stiffer and softer segments 12, 14 of the vaso-occlusive coil 10 may be modified using a variety of techniques. In one embodiment, the stiffness of the vaso-occlusive coil 10 is modified by varying several parameters during manufacture of the vaso-occlusive coil 10. As described above, vaso-occlusive coils 10 are formed by helically winding a filament 16 about a “primary” mandrel 30 to form the coil 10. As shown in FIG. 4, the filament 16 is fed to an elongate mandrel 30 from a carriage 32 traveling along a track 34, which can be used to adjust several winding parameters. The track 34 is parallel to the longitudinal axis of the elongate mandrel 30. The mandrel 30 is rotated by a motor (not shown) while the carriage 32 moves along the track 34 to helically wind the filament 16 into a coil 10. The carriage 32 moves in the direction of the “growing” coil 10. The rate of movement of the carriage 32 is proportional to the rate of rotation of the mandrel 30 and the width of the filament 16. More particularly, movement of the carriage 32, measured in widths of the filament 16 per second, is equal to rotation of the mandrel 30, measured in rotations per second. This relationship between carriage movement and mandrel rotation ensures a closed pitch coil 10 with substantially no gaps between windings, which minimizes spring action during delivery of the vaso-occlusive coil 10.

The mandrel 30 is an elongate body having first and second ends 36, 38. In one embodiment, the first and second ends 36, 38 of the mandrel 30 can be respectively held in a motor and an arbor by chucks. The distance between the motor and the arbor can be adjusted to fit various mandrels 30. A solenoid attached to one of the ends 36, 38 can exert axial/longitudinal tension on the mandrel 30. The carriage 32 includes a spool for holding the filament 16 and a pulley, which can be moved to change the tension on the filament 16.

The first winding parameter that can be varied to modify the stiffness of vaso-occlusive coil 10 is the tension on the filament 16 during winding. Filament tension is adjusted by a mechanism (e.g., the pulley described above) pulling on the filament 16 at the carriage/spool 32. Alternatively, filament tension can be adjusted by moving the mandrel 30 and the track 34 (and therefore the carriage 32) toward or away from each other in the plane formed by the track 34, the longitudinal axis of the mandrel 30 and the length of filament 16 extending between these two lines. Increasing filament tension increases stiffness of the resulting segment of coil 10. Decreasing filament tension decreases stiffness of the resulting segment of coil 10.

The second winding parameter that can be varied to modify the stiffness of vaso-occlusive coil 10 is the angle 40 of the filament 16 relative to leading edge of the growing coil 10 on the mandrel 30 during winding. Filament angle can be modified by adjusting the location of the carriage 32 relative to the leading edge of the growing coil 10. When the filament angle 40 is 0°, the filament 16 is perpendicular to the mandrel 30, as shown in FIG. 5. When the filament angle is positive, the carriage 32 “leads” the coil 10 in that the carriage 32 pulls the filament 16 ahead of the forming coil 10 on the mandrel 30. With a positive filament angle, there is a very small opening/gap between the filament 16 and the previous wind of the forming coil 10. Existing winding devices have limited ability to wind a positive angle as they adjust the horizontal travel of the carriage 32 on the track 34 to “catch up” and maintain relatively closed pitch. When the filament angle is negative, the carriage 32 “lags behind” the coil 10 in that the carriage 32 pulls the filament 16 in the opposite direction of the forming coil 10 on the mandrel 30. With a negative filament angle, the filament 16 is angled so it is in contact with the previous wind of the forming coil 10. Increasing the filament angle during winding decreases stiffness of the resulting segment of coil 10. Decreasing filament angle during winding increases stiffness of the resulting segment of coil 10. In various embodiments, the filament angle may vary from −90 degrees to +90 degrees.

The third winding parameter that can be varied to modify the stiffness of vaso-occlusive coil 10 is the axial/longitudinal tension on the elongate mandrel 30 during winding. Mandrel tension is adjusting by pulling the first and second ends 36, 38 of the mandrel 30 away from each other along the longitudinal axis of the mandrel 30 with varying amounts of force (e.g., using the solenoid described above). According to one theory, decreasing mandrel tension indirectly drives coil stiffness by increasing (1) the vibration of the mandrel 30 during winding and (2) the deflection of the mandrel 30 when filament tension is applied to the mandrel 30. These effects, in turn, reduce stability of the filament tension and angle winding, thereby further stressing the filament 30 and increasing coil stiffness. According to this theory, increasing mandrel tension decreases stiffness of the resulting segment of coil 10, and decreasing mandrel tension increases stiffness of the resulting segment of coil 10. Mandrel tension is a secondary winding parameter and does not affect coil segment stiffness as much as primary winding parameters (filament tension and filament angle).

These primary and secondary winding parameters (filament tension, filament angle, and mandrel tension) can be varied using existing coil winding devices. However, the winding parameters are currently only varied between coils 10 and not during the winding of a single coil 10. Nevertheless, varying winding parameters to wind a coil 10 with stiffer and softer segments 12, 14 can be implemented by reprogramming the software of existing coil winding devices.

According to various theories, varying winding parameters while winding coils 10 from filaments 16 imparts microstructural changes to the filament 16. According to one theory, winding coils with high stress winding parameters (e.g., high filament tension, and low (negative) filament angle) stresses or “cold works” the metal in the filament 16 (like a blacksmith using a hammer), thereby adding stress to and reducing the grain size of the metal. The grain size is reduced by limiting the growth of the grain structure through application of force to the metal. The reduced grain size increases the stiffness of the resulting segment of coil 10.

According to another theory, winding coils with high stress winding parameters deforms the grains leading to increased stiffness. Strain hardening/stressing/cold working the filament increases the dislocation density within the filament material by plastically deforming the material, resulting in atomic slip along slip planes and introduction of dislocations within grains. When in the same plane, these dislocations repel each other and provide another barrier to the slip plane of the crystals. This results in increased stiffness/strength/hardness of the material.

To demonstrate the effect of these primary and secondary winding parameters on coil stiffness, two coils 10 were wound using the winding parameters in Table 1:

TABLE 1

| Coil No. | Filament Tension | Mandrel Tension | Filament Angle |
|---|---|---|---|
| 1 | 10 g | 700 g | 5° |
| 2 | 40 g | 300 g | −1° |

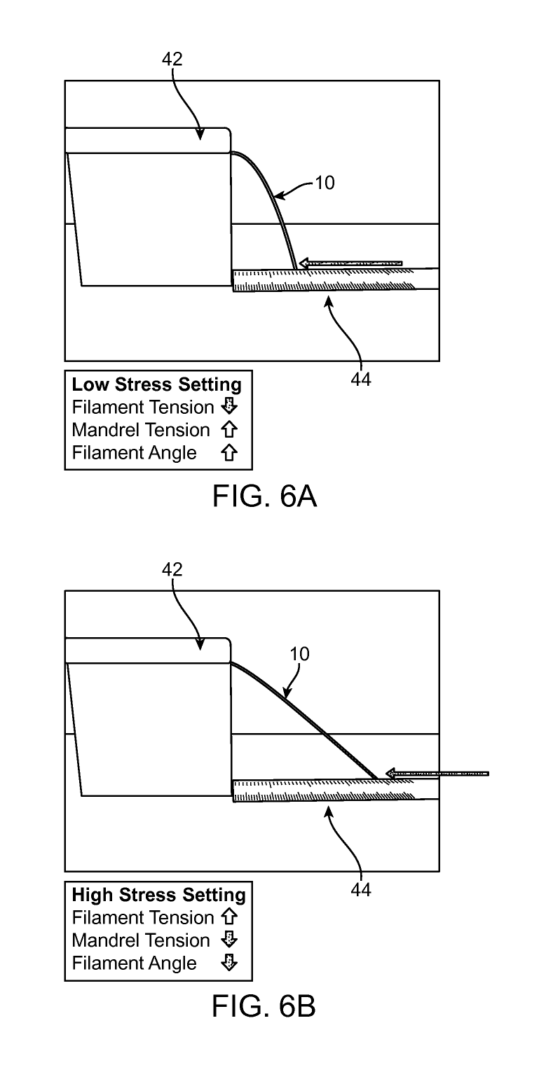

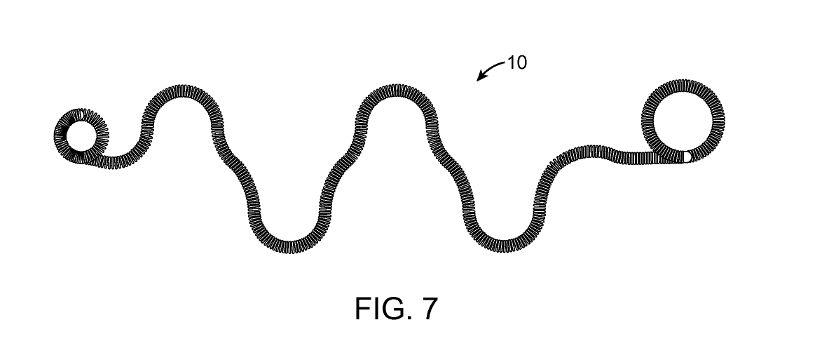

The resulting coils 10 are depicted in FIGS. 6A and 6B, with one portion of each coil 10 supported on a higher surface 42 and the remaining portion of each coil 10 unsupported and suspended over a lower surface 44 from an end of the (higher) supported portion. Coil No. 1, which was wound under low stress settings shown in FIG. 6A, forms an arcuate shape that approximates half of a hyperbola with the vertex of the hyperbola at the end of the supported portion of the coil 10. Coil No. 2, shown in FIG. 6B, forms an approximate straight line from the end of the supported portion to the lower surface 44. The difference shapes of the two coils 10 demonstrate that Coil No. 1 is less stiff (more flexible) than Coil No. 2, because the latter can maintain a substantially linear form when unsupported, but the former deforms into an arcuate shape under the weight of the unsupported portion of the coil 10. In fact, while Coil No. 1 and Coil No. 2 were wound from the same filament 16 with substantially similar pitch and outer diameter, the stiffness of Coil No. 2 (high stress winding, FIG. 6B) is approximately double that of Coil No. 1 (low stress winding, FIG. 6A). Comparing the vaso-occlusive coils 10 in FIGS. 6A and 6B demonstrates that imparting stress to (i.e., working) the filament 10 while winding affects performance of the resulting coil 10, including its modulus.

In other embodiments, the vaso-occlusive coil 10 can be stiffened by twisting the filament 16 during winding to stress the filament 16. For instance, the spool on the carriage 32 can be rotated during the winding process to twist the filament 16 as it is being wound. Increasing the rate of rotation will increase the twisting of the filament 16 and the stress in the filament 16, thereby winding stiffer coil segments 12.

In still other embodiments, the stiffness of vaso-occlusive coils 10 made by mandreless winding can also be modified by adjusting winding parameters during winding. Adjustable mandreless winding parameters include the amount of force with which the filament 10 is urged against the deflection surface, filament tension, filament angle, winding speed, winding pitch, and the shape of the contact point.

To further increase the relative stiffness/softness difference between the segments 12, 14 to increase coil versatility, small variations in pitch can be incorporated into the respective segments, while maintaining the substantially similar coil construction. More open pitched segments are softer than closed pitched segments. However, opening the pitch of vaso-occlusive coils 10 creates a spring effect that interferes with delivery.

Moreover, the relative stiffness/softness difference between the segments 12, 14 can be increased by heating segments of the coil 10. The coil 10 can be selectively heat treated by wrapping around a ferromagnetic mandrel and heating inductively with a fluctuating magnetic field. The mandrel can be made with a combination of ferromagnetic steel and non-magnetic material. Accordingly, only the ferromagnetic steel portions of the mandrel and the segments 14 of the coil 10 wrapped around those portions of the mandrel will be heated. Heating metals increases the grain size of the metal, making it softer. Therefore, these heat treated segments 14 will be softened by the heat treatment. The segments 14 may be selected such that the segments 14 will promote earlier coil complexion when deployed into an aneurysm (i.e., the coil changing from one orthogonal plane to another in three dimensional space). Alternatively, the distal portion of the coil 10 may be made “softer” through heat treating while the proximal portion remains in the original secondary shape heat-treated condition to provide superior retention strength. For instance, by heating a coil 10 around a mandrel, a regular sized GDC-18-3D coil can be made to have the softness of a 10 type coil. Such coils 10 may also be made with the stretch resistant design if desired and have lower friction than regular three dimensional coils. Alternatives to inductive heating using a ferromagnetic mandrel include resistive and laser heating of segments of the coil 10.

In any of the embodiments described herein, the filament 16 may have a cross-sectional dimension that is in the range of 0.00002 and 0.01 inches. The windings may have a cross-sectional dimension between 0.003 and 0.03 inches. In various embodiments, the filament 16 can have any geometry/shape, such as triangle, rectangle, square, or circle. For neurovascular applications, the diameter of the windings may be anywhere from 0.008 to 0.025 inches. In other embodiments, the filaments 16 may have other cross-sectional dimensions, and the windings may have other cross-sectional dimensions. In some embodiments, the filament 16 for forming the windings should have a sufficient diameter to provide a hoop strength to the resulting vaso-occlusive coil 10 sufficient to hold the coil 10 in place within the chosen body site, lumen or cavity, without substantially distending the wall of the site and without moving from the site as a result of the repetitive fluid pulsing found in the vascular system.

In any of the embodiments described herein, the axial length of the vaso-occlusive coil 10 may be in the range of 0.5 mm to 100 cm, and more preferably, in the range of 1.0 to 65 cm. Depending upon use, the vaso-occlusive coils 10 may have 10-75 turns per millimeter, or more preferably 10-40 turns per millimeter. In other embodiments, the vaso-occlusive coils 10 may have other lengths and/or other number of turns per millimeter.

Further, while the above-described embodiments are directed to single layer vaso-occlusive coils 10, it should be appreciated by those skilled in the art that double-coil embodiments, i.e., vaso-occlusive coils 10 having an outer coil layer and an inner coil layer may be included in alternative embodiments, in accordance with the features of the embodiments disclosed herein.

In some embodiments, the vaso-occlusive devices 10 described herein may have a minimal secondary shape in addition to the three dimensional shape resulting from the configuration of stiffer and softer segments 12, 14. Such minimum secondary shape features include a preset shape on the distal end, such as “J” or 45 degree angle, to direct the coil more centrally, and a helical loop on proximal end to prevent the coil tail from extending from the aneurysm into the parent vessel. Such vaso-occlusive devices 10 would have shapes that are more complex. FIG. 6 shows what is termed a “secondary” shape in that it is formed from the primary coil by winding the primary coil on a form of a desired shape, e.g. a mandrel, and then heat treating the so-formed shape. Various other secondary shapes may be implemented in embodiments of the vaso-occlusive devices 10 described herein.

While the illustrated and described embodiments have segments of two relative stiffnesses (i.e., stiffer and softer segments 12, 14), other embodiments include vaso-occlusive coils 10 that have three or more different stiffnesses disposed along the length of the coil 10. For instance, a distal most first segment of the coil 10 can have the greatest softness, a second segment proximally adjacent to the first segment can have a moderate amount of softness/stiffness, and a third segment proximally adjacent to the second segment can have the least softness.

Further, while the lengths of respective stiffer and softer segments 12, 14 in the illustrated and described embodiments are constant throughout the length of the vaso-occlusive coil 10, the lengths of these segments 12, 14 can vary in different sections of the coil 10. For instance, the distal portion of the coil 10 can have segments 12, 14 of longer length for seeking an open space in and framing an aneurysm in the early stages of coil 10 delivery. The proximal portion of the coil 10 can have segments 12, 14 of shorter length for filling the framed aneurysm in the later stages of coil 10 delivery. The respective segment length can also be made to gradually change along the length of the coil 10.

For instance, the respective lengths of the stiffer segments 12 between the distal and proximal portions of the coil 10 can gradually decrease from a longer length (e.g., 5 mm) to a shorter length (e.g. 0.2 mm). Decreasing segment lengths from the distal portion of the coil 10 to the proximal portion of the coil 10 results in more frequent bending of the coil as the proximal portion is ejected from the delivery catheter after the distal portion has been ejected. Accordingly, the distal portion of the coil 10, which is deployed first, will frame the aneurysm with larger segments between bends, and the proximal portion of the coil 10, which is deployed last, will fill the framed aneurysm with smaller segments and more frequent bends.

Moreover, while the illustrated and described embodiments include multiple softer segments 14 alternating between multiple stiffer segments 12, other embodiments (e.g., in a relatively short or finishing coil) may include only a single softer segment 12 that divides the remainder of the coil into two stiffer segments 14, resulting in only a single bending location in the coil.

Although particular embodiments have been shown and described herein, it will be understood by those skilled in the art that they are not intended to limit the present inventions, and it will be obvious to those skilled in the art that various changes and modifications may be made (e.g., the dimensions of various parts) without departing from the scope of the disclosed inventions, which is to be defined only by the following claims and their equivalents. The specification and drawings are, accordingly, to be regarded in an illustrative rather than restrictive sense. The various embodiments shown and described herein are intended to cover alternatives, modifications, and equivalents of the disclosed inventions, which may be included within the scope of the appended claims.

What is claimed is:

1. A vaso-occlusive device, comprising:
an elongate strand of material wound by a winding process into a helical coil having a length,
wherein one or more sections of the coil along its length were subjected to less mechanical stress or strain than a remainder of the coil during the winding process,
such that each of the one or more sections has a lower stiffness and greater flexibility than a respective stiffness and flexibility of the remainder of the coil, and
such that each of the one or more sections acts as a bending location along the length of the coil when the coil is delivered into an aneurysm.

2. The vaso-occlusive device of claim 1, wherein the one or more sections of the coil were subjected to less mechanical stress or strain than the remainder of the coil due to a temporary decrease in a tension imparted on the elongate strand during winding of the respective one or more sections.

3. The vaso-occlusive device of claim 1, wherein the one or more sections of the coil were subjected to less mechanical stress or strain than the remainder of the coil due to a temporary increase in an angle formed by the elongate strand and a mandrel on which the coil was wound during winding of the respective one or more sections.

4. The vaso-occlusive device of claim 1, wherein the one or more sections of the coil have substantially same dimensions, including a substantially same pitch, as windings of the remainder of the coil.

5. The vaso-occlusive device of claim 1, the one or more sections consisting of a single bending section along the length of the coil.

6. The vaso-occlusive device of claim 1, the one or more sections comprising a plurality of bending sections along the length of the coil.

7. The vaso-occlusive device of claim 6, the bending sections being substantially uniform in length.

8. The vaso-occlusive device of claim 6, at least one bending section having a length different than a respective length of one or more other bending sections of the plurality.

9. A vaso-occlusive device, comprising:
an elongate strand of material wound by a winding process into a helical coil, the coil comprising a plurality of bending sections and a plurality of non-bending sections, wherein individual bending sections alternate with individual non-bending sections along a length of the coil, and
wherein the bending sections were subjected to less mechanical stress or strain than the non-bending sections during the winding process, such that the bending sections have a lower stiffness and greater flexibility than a respective stiffness and flexibility of the non-bending sections.

10. The vaso-occlusive device of claim 9, wherein the bending sections were subjected to less mechanical stress or strain than the non-bending sections during the winding process due to a temporary decrease in a tension imparted on the elongate strand during winding of each bending section.

11. The vaso-occlusive device of claim 9, wherein the bending sections were subjected to less mechanical stress or strain than the non-bending sections during the winding process due to a temporary increase in an angle formed by the elongate strand and a mandrel on which the coil was wound during winding of each bending section.

12. The vaso-occlusive device of claim 9, wherein the bending sections have substantially same dimensions, including a substantially same pitch, as windings of the non-bending sections.

13. The vaso-occlusive device of claim 9, the bending sections being substantially uniform in length.

14. The vaso-occlusive device of claim 9, at least one bending section having a length different than a respective length of one or more of the other bending sections.

15. The vaso-occlusive device of claim 9, the non-bending sections being substantially uniform in length.

16. The vaso-occlusive device of claim 9, at least one non-bending section having a length different than a respective length of one or more of the other non-bending sections.

17. The vaso-occlusive device of claim 9, the non-bending sections decreasing in length from a distal end of the coil to a proximal end of the coil.

18. The vaso-occlusive device of claim 9, wherein at least some transitions between respective non-bending sections and bending sections are gradual.

19. The vaso-occlusive device of claim 9, wherein at least some transitions between respective non-bending sections and bending sections are abrupt.

20. A vaso-occlusive device, comprising:
an elongate strand of material wound by a winding process into a helical coil, the coil comprising a plurality of bending sections and a plurality of non-bending sections, wherein individual bending sections alternate with individual non-bending sections along a length of the coil, and
wherein the bending sections were subjected to less mechanical stress or strain than the non-bending sections during the winding process due to one or both of (i) a temporary decrease in a tension imparted on the elongate strand during winding of each bending section, and (ii) a temporary increase in an angle formed by the elongate strand and a mandrel on which the coil was wound during winding of each bending section,
such that the bending sections have a lower stiffness and greater flexibility than a respective stiffness and flexibility of the non-bending sections.
